(19) United States
(12) Patent Application Publication
(10) Pub. No.: US 2025/0288269 A1
(43) Pub. Date: Sep. 18, 2025

(54) DYNAMIC ANALYSIS APPARATUS, DYNAMIC ANALYSIS METHOD, AND STORAGE MEDIUM

(71) Applicant: Konica Minolta, Inc., Tokyo (JP)

(72) Inventor: Noritsugu MATSUTANI, Tokyo (JP)

(21) Appl. No.: 19/080,377

(22) Filed: Mar. 14, 2025

(30) Foreign Application Priority Data
Mar. 18, 2024 (JP) 2024-041870

(51) Int. Cl. A61B 6/50; A61B 6/00; G06T 7/00; G06T 7/11; G06T 11/00

(52) U.S. Cl. CPC A61B 6/504; A61B 6/5205; G06T 7/0012; G06T 7/11; G06T 11/001; G06T 2207/30061; G06T 2207/30104

(57) ABSTRACT

A dynamic analysis apparatus performs dynamic analysis on a dynamic image obtained by irradiating a subject with radiation and includes a hardware processor. The hardware processor generates a dynamic analysis image for measuring a blood flow, based on signal values of pixels constituting the dynamic image. When a pixel of the dynamic analysis image has a signal value less than or equal to a first threshold, the hardware processor detects the pixel as a signal decrease region that indicates a decrease in a blood flow value. The hardware processor compares a quantitative value of the detected signal decrease region or a quantitative value of a non-signal decrease region with a second threshold, the non-signal decrease region being other than the signal decrease region, and based on the comparison, determines whether a blood flow defect is present.

### CROSS-REFERENCE TO RELATED APPLICATIONS

The entire disclosure of Japanese Patent Application No. 2024-041870 filed on Mar. 18, 2024, is incorporated herein by reference in its entirety.

### BACKGROUND OF THE INVENTION

### Technical Field

The present invention relates to a dynamic analysis apparatus, a dynamic analysis method, and a storage medium.

### Description of Related Art

As a type of analysis processing on a dynamic image, there has been developed analysis processing capable of visualizing the amount of change in high-frequency signals in a lung field synchronized with a heartbeat and expressing a minute change in a blood flow rate. Such analysis processing is effective in evaluating pulmonary embolism, for example. Japanese Unexamined Patent Publication No. 2022-149901 describes a dynamic analysis apparatus that adds colors to pixels of a dynamic analysis result according to their signal values to display a colored or binarized dynamic analysis result.

According to a known technology, changes in the amount of blood flow in the lung field are expressed in color or in a binary manner. A physician unskilled in image reading cannot correctly determine the presence of a blood flow defect unless the physician is trained in image reading. Further, the degree of a blood flow defect cannot be accurately evaluated in some cases because image interpretation is subjective.

In order to solve the above-described problem, an object of the present invention is to provide a dynamic analysis apparatus, a dynamic analysis method, and a program that allow a doctor unskilled at interpretation of a dynamic analysis image to accurately perform diagnosis.

### SUMMARY OF THE INVENTION

To achieve at least one of the abovementioned objects, according to an aspect of the present invention, a dynamic analysis apparatus reflecting one aspect of the present invention is configured to perform dynamic analysis on a dynamic image obtained by irradiating a subject with radiation and includes a hardware processor, wherein: the hardware processor generates a dynamic analysis image for measuring a blood flow, based on signal values of pixels constituting the dynamic image; when a pixel of the dynamic analysis image has a signal value less than or equal to a first threshold, the hardware processor detects the pixel as a signal decrease region that indicates a decrease in a blood flow value; and the hardware processor compares a quantitative value of the detected signal decrease region or a quantitative value of a non-signal decrease region with a second threshold, the non-signal decrease region being other than the signal decrease region, and based on the comparison, determines whether a blood flow defect is present.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is an example of a schematic configuration of a radiographing imaging system according to an embodiment;

FIG. 2 is an example of a block diagram of a dynamic analysis apparatus according to the present embodiment;

FIG. 3 is a flowchart showing an example of operation of the dynamic analysis apparatus in determining the presence of a blood flow defect, based on a PH2-mode dynamic analysis image according to the present embodiment;

FIG. 4 is a flowchart showing the flow of a PH2-mode dynamic analysis process according to the present embodiment;

FIG. 5 is a diagram illustrating a PH2-mode dynamic analysis image colored according to the present embodiment;

FIG. 6 is a diagram illustrating an example of a dynamic analysis image colored based on the levels of signal values of the respective pixels after gain adjustment processing according to the present embodiment;

FIG. 7 is a diagram illustrating an example of a dynamic analysis image after emphasis processing is performed on a signal decrease region according to the present embodiment;

FIG. 8 is a diagram illustrating an example of a PH2-mode dynamic analysis image, a dynamic analysis image indicating a signal decrease region, and statistical information displayed on a display according to the present embodiment;

FIG. 9 is a diagram illustrating an example of a method of setting a first threshold and so forth according to a first modification example; and

FIG. 10 is a diagram illustrating an example of a method for setting a first threshold and so forth according to a second modification example.

### DETAILED DESCRIPTION

Hereinafter, one or more embodiments of the present invention will be described with reference to the drawings. However, the scope of the invention is not limited to the disclosed embodiments.

In the following, a preferred embodiment of the present disclosure will be described in detail with reference to the accompanying drawings.

### Configuration Example of Radiographic Imaging System 100

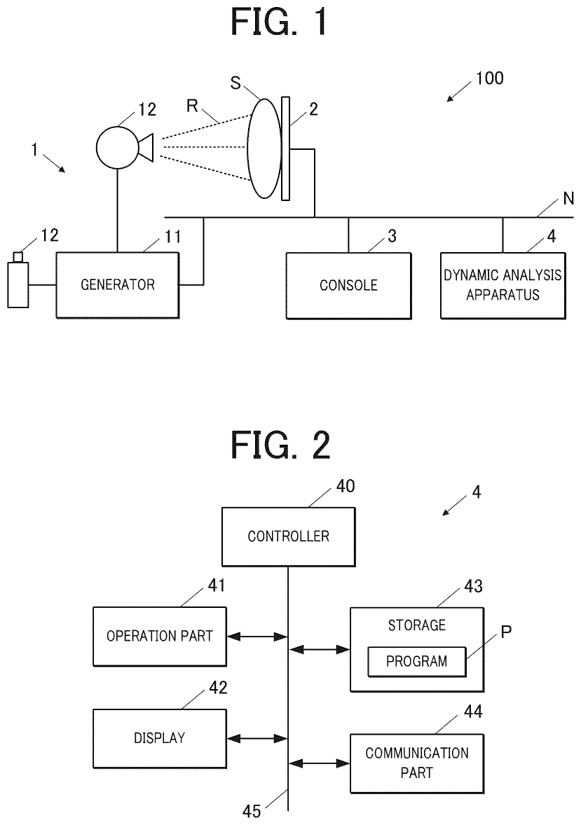

FIG. 1 is a diagram illustrating an example of a schematic configuration of a radiographic imaging system 100 according to the present embodiment. The radiographic imaging system 100 includes a radiation generating apparatus 1, a radiographic imaging apparatus 2, a console 3, and a dynamic analysis apparatus 4. Hereinafter, the radiation generating apparatus 1 may be referred to as a generating apparatus 1, the radiographic imaging apparatus 2 may be referred to as an imaging apparatus 2. The generating apparatus 1, the imaging apparatus 2, the console 3, and the dynamic analysis apparatus 4 are communicably connected to each other via a network N. Examples of the network N include a LAN, a WAN, and the Internet. LAN is an abbreviation for Local Area Network. WAN is an abbreviation for Wide Area Network. A communication system of the network N may be wired communication or wireless communication.

The generating apparatus 1 includes a generating apparatus 11, an exposure switch 12, and a radiation source 13. The generating apparatus 11 applies, to the radiation source 13 including, for example, a tube, a voltage corresponding to preset imaging conditions in response to operation of the exposure switch 12. The generating apparatus 11 may include an operation receiver that receives input of irradiation conditions and the like. When a voltage is applied by the generating apparatus 11, the radiation source 13 generates radiation R having a dose corresponding to the applied voltage. The radiation R is, for example, X-rays.

The generating apparatus 1 generates the radiation R in a manner corresponding to the type of radiation image, for example, a still image or a dynamic image. Specifically, to capture a still image, the generating apparatus 1 emits the radiation R only once in response to the exposure switch 12 being pressed once. To capture a dynamic image, the generating apparatus 1 acquires a series of images of the subject S in response to one time of imaging operation by dynamic imaging. In dynamic imaging, the subject S is repeatedly irradiated with pulsed radiation, such as X-rays, at predetermined time intervals. Repeatedly applying pulsed radiation at predetermined time intervals is referred to as pulsed irradiation. The dynamic imaging includes obtaining a series of images of the subject S by continuously irradiating the subject S at a low dose rate in response to one imaging operation. Continuously applying radiation without interruption is referred to as continuous irradiation. A series of images obtained by dynamic imaging is called a dynamic image. Images constituting a dynamic image are called frame images. The dynamic imaging includes moving image capturing but does not include capturing of a still image while displaying a moving image.

Further, examples of a dynamic image include a moving image but do not include still images captured while displaying a moving image.

The imaging apparatus 2 generates digital image data in which an imaging part of the subject S is captured. For example, a portable FPD is used as the imaging apparatus 2. FPD is an abbreviation for Flat Panel Detector. The imaging apparatus 2 may be integrated with the generating apparatus 1.

Although not illustrated, the imaging apparatus 2 includes, for example, an imaging element, a sensor substrate, a scanner, a reader, a controller, and a communication section. The imaging element generates electric charges according to the dose of received radiation R. In the sensor substrate, switch elements are arranged two-dimensionally (in a matrix), and in a two-dimensional array (matrix), and accumulate and discharge the electric charges. The scanner switches on and off of each switch element. The reader reads, as a signal value, an amount of electric charge emitted from each pixel. The controller generates image data of the radiation image from the plurality of signal values read by the reader. The image data includes still image data or dynamic image data. The communication section transmits the generated image data, various signals, and the like to other apparatuses, such as the console 3, and receives various kinds of information and signals from other apparatuses.

The console 3 sets imaging conditions for the generating apparatus 1, the imaging apparatus 2, and the like, and controls a reading operation of radiographic images obtained by the imaging apparatus 2. The console 3 is also referred to as an imaging control apparatus and is constituted by, for example, a personal computer.

The imaging conditions include, for example, patient conditions related to the subject S, irradiation conditions related to the emission of the radiation R, and image reading conditions related to the image reading of the imaging apparatus 2. The patient conditions include, for example, an imaging site, an imaging direction, and a physique. The irradiation conditions are, for example, tube voltage (kV), tube current (mA), irradiation time (ms), current-time product (mAs value), and the like. The image reading conditions include, for example, a pixel size, an image size, and a frame rate. The console 3 may automatically set the imaging conditions, based on order information acquired from a HIS/RIS, for example. The console 3 may set the imaging conditions in response to manual operations by a user, such as a doctor or a radiologist, on an operation part 41 described later.

The dynamic analysis apparatus 4 performs dynamic analysis on dynamic images acquired from other apparatuses, such as the imaging apparatus 2 and the console 3. For example, as one type of dynamic analysis, the dynamic analysis apparatus 4 visualizes the amount of change (change in blood flow volume) of high-frequency signals in the lung field synchronizing with the heartbeat and executes processing to express minute changes in the amount of blood flow. In the present embodiment, this dynamic analysis process is referred to as a PH2 mode. Specifically, the dynamic analysis apparatus 4 calculates, as the blood flow feature quantity of each pixel, a difference between a signal value of a pixel in a predetermined region extracted from the reference frame of the dynamic image and a signal value of a pixel in a corresponding predetermined region of another frame image. The dynamic analysis apparatus 4 displays the dynamic analysis result of the dynamic image on the screen of the display part 42.

### Configuration Example of Dynamic Analysis Apparatus 4

Next, the configuration of the dynamic analysis apparatus 4 according to the present embodiment will be described. FIG. 2 is an example of a block diagram of the dynamic analysis apparatus 4 according to the present embodiment.

The dynamic analysis apparatus 4 includes a controller 40 (hardware processor), the operation part 41, the display part 42, a storage section 43, and a communication section 44. The controller 40, the operation part 41, the display part 42, the storage section 43, and the communication section 44 are connected to each other via wires, such as a bus 45.

The controller 40 includes, for example, a processor, such as a CPU that performs calculations and control, and a memory. CPU is an abbreviation for Central Processing Unit. The controller 40 executes, for example, a program P (described later) stored in a memory, such as a RAM or the storage section 43, to perform a dynamic analysis process with a dynamic image, a determination process, and so forth. In the determination process, the controller 40 determines whether a blood flow defect or the like is present, based on a dynamic analysis image. The controller 40 may include an electronic circuit, such as an ASIC or an FPGA. ASIC is an abbreviation of Application Specific Integrated Circuit. FPGA is an abbreviation for Field Programmable Gate Array.

In the present embodiment, the controller 40 functions as a generation unit, a correction unit, a detection unit, a determination unit, and an output unit. The processor of the controller 40 performs the functions of the generation unit, the correction unit, the detection unit, the determination unit, and the output unit by executing the program P stored in the storage section 43 or the like. The generation unit generates a dynamic analysis image for measuring a blood flow rate, based on signal values of pixels of the dynamic image. Specifically, as the dynamic analysis process, the generation unit calculates a difference between a signal value of each pixel of one reference frame constituting the dynamic image and a signal value of each corresponding pixel of other frames and generates a PH2-mode dynamic analysis image. The correction unit improves the contrast of the dynamic analysis image by correcting variations in generated dynamic analysis images caused by different imaging conditions for different times of imaging. When a signal value of each pixel of the dynamic analysis image is equal to or less than a first threshold, the detection unit detects the pixel as a signal decrease region that indicates a decrease of a blood flow value. The first threshold is for determining whether each pixel of the dynamic analysis image is a signal decrease region indicating a decrease of blood flow. The determination unit determines whether a blood flow defect is present, based on comparison between a quantitative value (e.g., the area) of the detected signal decrease region or a non-signal decrease region other than the signal decrease region and a second threshold. When the quantitative value is an area, the second threshold is for determining whether the area of the signal decrease region corresponds to a blood flow defect. When it is determined that a blood flow defect is present in the dynamic image, the output unit outputs determination information regarding the blood flow defect to the display part 42 or the like.

The operation part 41 includes, for example, a mouse, a keyboard, a switch, and a button. The operation part 41 may be, for example, a touch screen integrally combined with a display or may be an interface that receives a voice input. The operation part 41 receives a command according to various input operations from a user, converts the received command into an operation signal, and outputs the operation signal to the controller 40.

The display part 42 is, for example, a display such as a liquid crystal display or an organic EL display. EL is an abbreviation for Electro Luminescence. The display part 42 displays an image on which predetermined analysis processing has been performed, a GUI for receiving various input operations by a user, and so forth. GUI is an abbreviation for Graphical User Interface. Specifically, the display part 42 displays a dynamic analysis image obtained by dynamic analysis on the dynamic image and determination information including the determination on whether a blood flow defect is present, based on the dynamic analysis image.

The storage section 43 includes, for example, a storage module such as an HDD, an SSD, a ROM, and a RAM. HDD is an abbreviation of Hard Disk Drive. SSD is an abbreviation for Solid State Drive. ROM is an abbreviation of Read Only Memory. The storage section 43 stores, for example, a system program, an application program, and various types of data. Specifically, the storage section 43 stores a program P for executing the dynamic analysis process of a dynamic image, the determination process for determining whether a blood flow defect or the like is present, based on a dynamic analysis image, and so forth. The storage section 43 may store the first threshold and the second threshold. The first threshold is for determining whether each pixel of the dynamic analysis image corresponds to the signal decrease region indicating a decrease in blood flow. The second threshold is for determining whether the area of the signal decrease region corresponds to a blood flow defect or the like.

The communication part 44 includes, for example, a communication module including an NIC, a receiver, and a transmitter. NIC is an abbreviation for Network Interface Card. The communication part 44 sends and received various information and image data to and from the generating apparatus 1, the imaging apparatus 2, the console 3, and the like via the network N.

### Operation Example of Dynamic Analysis Apparatus 4

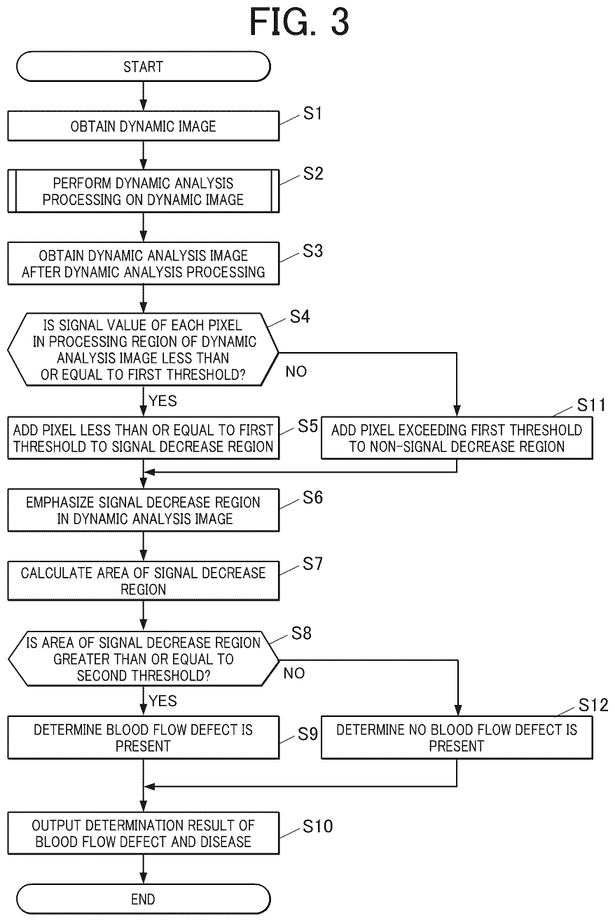

Next, a flow of the dynamic analysis process method according to the present embodiment will be described. FIG. 3 is a flowchart showing an example of operation of the dynamic analysis apparatus 4 in determining the presence of a blood flow defect, based on a PH2-mode dynamic analysis image according to the present embodiment. Following is a case where the processing region in the dynamic image is a lung field region. As the dynamic analysis process, a PH2-mode process is performed. In the PH2 mode, the amount of change in high-frequency signals in the lung field synchronizing with the heartbeat is visualized, and a minute change in the amount of blood flow is expressed.

The controller 40 acquires a dynamic image from another apparatus, such as the imaging apparatus 2 or the console 3, via the communication part 44 (step S1). After acquiring the dynamic image, the controller 40 proceeds to step S2.

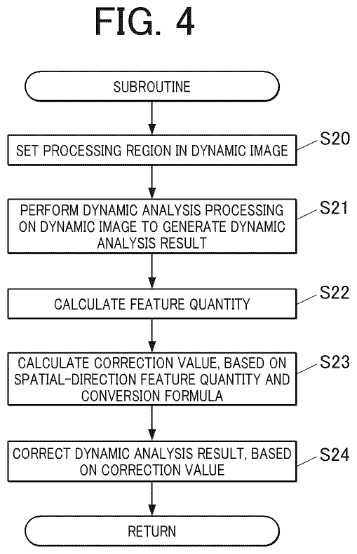

The controller 40 performs the PH2-mode dynamic analysis process on the acquired dynamic image (step S2). The controller 40 proceeds to the subroutine illustrated in FIG. 4 to perform the PH2-mode dynamic analysis process. FIG. 4 is a flowchart showing the flow of a PH2-mode dynamic analysis process according to the present embodiment. To perform the PH2-mode dynamic analysis process, a technique disclosed in Japanese Unexamined Patent Publication can be applied.

As illustrated in FIG. 4, the controller 40 sets an analysis region RA in each frame of the acquired dynamic image (step S20). The analysis region RA coincides with a region including the processing region RP that is set in the processing region setting process, which will be described later. The analysis region RA includes the lung field region. The analysis region RA may be acquired, for example, by using a learned model that has been trained with machine learning to output the analysis region RA for the acquired dynamic image. In such a case, the controller 40 inputs a dynamic image showing the lung field region to the learned model and acquires a dynamic image having the analysis region R from the learned model, as the output result. On the dynamic image having the analysis region R, multiple contour points are added on the contour line of the lung field region. The analysis region RA may be set by any other automatic method or may be manually set by the user.

The controller 40 divides the analysis region RA, which is set on each frame constituting the dynamic image, into multiple block regions RB each having a size of one pixel or more. For example, the controller 40 divides the analysis region RA into 10 mm×10 mm rectangular block regions RB arranged in a matrix. After setting the analysis region, the controller 40 proceeds to step S21.

The controller 40 generates a PH2-mode dynamic analysis image G1 by performing dynamic analysis on the dynamic image (step S21). Step S21 corresponds to a generating step. For example, the controller 40 calculates a difference between the signal value of a pixel in one reference frame constituting the dynamic image and the signal value of a pixel in a different frame. The controller 40 may set different frames as the reference frame for the respective block regions RB. The reference frame may be, for example, a frame indicating the lowest signal value or a frame indicating the highest signal value in an RPI (heart region), which are set beforehand, among the frames. Although the dynamic analysis image G1 is a dynamic image, the dynamic analysis image G1 may be a still image or correlated values.

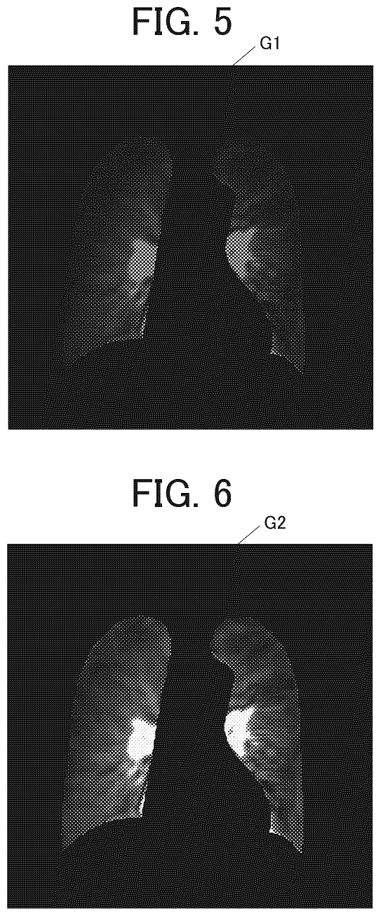

Regarding a patient with less blood flow, the amplitudes of the signal waveforms in the dynamic analysis image G1 are small, and the contrast is low when the pixels are displayed in color. FIG. 5 is the dynamic analysis image G1 that is acquired in step S21 in the present embodiment and colored. If the patient is short in stature, the blood flow is low. In such a case, the dynamic analysis image G1 shown in FIG. 5 has a lower color contrast, and a doctor may not correctly determine whether a blood flow defect is present. In the present embodiment, gain adjustment is performed on the generated dynamic analysis image G1 to improve the color contrast of the dynamic analysis image G1. When the dynamic analysis image G1 is generated, the controller 40 proceeds to step S22.

The controller 40 executes a feature amount calculation process (step S22). The feature amount calculation process includes a processing region setting process, a process for calculating a time-direction feature quantity, and a process for calculating a spatial-direction feature quantity. First, the controller 40 executes the processing region setting process to set a processing region RP in the dynamic image. For example, assume a case where the subject S is the chest of the subject and the dynamic analysis image G1 shows the analysis result of pulmonary blood flow. In the case, the controller 40 determines the processing region RP to be a pulmonary field region, a pulmonary hilum region, a cardiac region, an aortic arch region, and/or an arterial region.

Next, the controller 40 executes the process of calculating a time-direction feature quantity. For each pixel of the dynamic analysis image G1, the controller 40 calculates the maximum, the median, the minimum, the integrated value, and/or the mean value of the temporal change of the signal value, as the time-direction feature quantity. For example, the controller 40 determines time-direction feature quantity to be the minimum value of the temporal change of the signal value. The controller 40 creates a summarized image IS in which the calculated time-direction feature quantity of each pixel is aggregated.

Next, the controller 40 executes the process of calculating a spatial-direction feature quantity. From the summarized image IS obtained in the time-direction feature quantity calculation process, the controller 40 calculates a maximum value, a median value, a minimum value, an integrated value, or an average value as a spatial-direction feature quantity. For example, the controller 40 sets the minimum value of the time-direction feature quantities as the spatial-direction feature quantity. When the feature quantities are calculated, the controller 40 proceeds to step S23.

The controller 40 executes the correction value calculation process (step S23). The controller 40 calculates a correction value, based on the spatial-direction feature quantity calculated in the spatial-direction feature quantity calculation process and the conversion formula stored in the storage section 43. The conversion formula is for correcting the dynamic analysis image G1. The conversion formula may be determined, based on dynamic analysis images obtained by performing dynamic analysis on at least two dynamic images. Specifically, the conversion formula is obtained by linearly approximating a distribution of a scatter diagram that plots the relationship between (i) spatial-direction feature quantities obtained by different times of dynamic imaging and (ii) threshold values (gain values) obtained by digitizing the visually-checked actual blood flow of sites corresponding to the pixels of the spatial-direction feature quantities. The conversion formula is expressed as in the following (1).

Threshold=constant a×spatial-direction feature quantity−constant b . . . (1)

The controller 40 calculates the threshold by substituting the spatial-direction feature quantity, which is calculated in the feature-quantity calculation process, into the conversion formula (1). The controller 40 determines the correction value to be a multiplier required for obtaining the calculated threshold (a target value). In other words, the correction value is a value obtained by dividing the target value by the threshold. The conversion formula may not be stored in advance but may be newly calculated. The conversion formula may be updated, depending on a newly acquired dynamic image or the like. After the correction value is calculated, the controller 40 proceeds to step S24.

The controller 40 corrects the dynamic analysis image G1, based on the correction values calculated in the correction value calculation process (step S24). Specifically, the controller 40 multiplies the dynamic analysis image G1, such as difference values, evaluation values, and correlated values calculated for the respective pixels, by the correction value. The dynamic analysis image G1 after the multiplication is referred to as a corrected dynamic analysis image G2. By the correction process, the controller 40 can correct variations in the generated dynamic analysis images G1 caused by differences in imaging conditions between imaging operations. The controller 40 may correct the summarized image IS, which is created in the time-direction feature quantity calculation process, based on the correction value.

The controller 40 colors the pixels of the dynamic analysis image G2 after gain adjustment. Specifically, the controller 40 colors the pixels in the lung field region of the acquired corrected dynamic analysis image G2 according to levels of the signal values of the respective pixels. The pixels may be colored according to a table that associates signal values of pixels with levels of RGB, for example. In the present embodiment, the dynamic analysis image G2 after the correction is colored by gradations of 511 stages from black (R:0, G:0, B:0) to red (R:255, G:0, B:0) to yellow (R:255, G:255, B:0). A pixel with a less blood flow is colored with darker black, and a pixel with a greater blood flow is colored in red or yellow.

FIG. 6 is a diagram illustrating an example of the dynamic analysis image G2 that is colored based on the levels of signal values of the respective pixels after gain adjustment according to the present embodiment. In the dynamic analysis image G2, a region closer to the heart has a greater blood flow and higher signal values, so that the region is expressed in yellow. In FIG. 6, yellow is represented by white for convenience. On the other hand, a region distant from the heart is expressed in black because the blood flow is less, and the signal values are low. Further, in the dynamic analysis image G2, a disease region in which a disease, such as a blood flow defect, occurs has a less blood flow and low signal values, so that the disease region is expressed in black. By the gain adjustment, the color contrast can be increased for a patient with a less blood flow, so that a doctor can determine whether a blood flow defect is present relatively easily. When the coloring of the corrected dynamic analysis image ends, the controller 40 ends the subroutine and proceeds to step S3 in FIG. 3.

As illustrated in FIG. 3, the controller 40 classifies the pixels constituting the lung field region of the acquired dynamic analysis image G2 into (i) pixels having signal values equal to or less than the first threshold and (ii) pixels having signal values greater than the first threshold (step S4). That is, the controller 40 extracts pixels having signal values equal to or less than the first threshold from the pixels constituting the lung field region. Step S4 corresponds to a detection step. The first threshold as a reference can be determined to be a red tone close to black, which indicates a decreased blood flow and a high possibility of a blood flow defect. The red tone close to black corresponds to, for example, 15% of 511 tones of color, and corresponds to (R:76, G:0, B:0) in RGB. The correspondence between the occurrence of a blood flow defect and signal values of pixels constituting the dynamic image may be obtained, based on diagnostic results of past dynamic images, for example. Although the dynamic analysis image G2 is colored in 511 tones, the dynamic analysis image G2 may be colored in 0 to 100 tones, namely in 101 tones. In this case, 15 tones can be set as the first threshold indicating a blood flow defect. The first threshold is not limited to 15% of all tones of color but may be appropriately adjusted according to the purpose of the inspection. For example, since a screening inspection requires a higher level of sensitivity, the first threshold can be set to a smaller value.

The controller 40 adds pixels of the dynamic analysis image G2 that have been classified as having signal values equal to or less than the first threshold to the signal decrease region (step S5). The signal decrease region indicates that the blood flow is less than a reference value. After adding the pixels to the signal decrease region, the controller 40 proceeds to step S6.

The controller 40 adds pixels of the dynamic analysis image G2 that have been classified as having signal values greater than the first threshold to the non-signal decrease region (step S11). The non-signal decrease region indicates that the blood flow is greater than the reference value. In the dynamic analysis image G2, a region other than the signal decrease region corresponds to the non-signal decrease region. After adding the pixels to the non-signal decrease region, the controller 40 proceeds to step S6.

The controller 40 generates a dynamic analysis image G3 by performing emphasis processing on each pixel corresponding to the signal decrease region in the dynamic analysis image G2. Specifically, the controller 40 colors the pixels of the signal decrease region with a color corresponding to the signal decrease region. The color corresponding to the signal decrease region is different from the colors used in FIG. 6. The color of the signal decrease region is not limited to a specific color but may be, for example, yellowish green. The controller 40 does not add a new color to the pixels added to the non-signal decrease region, and maintains the color applied in step S24.

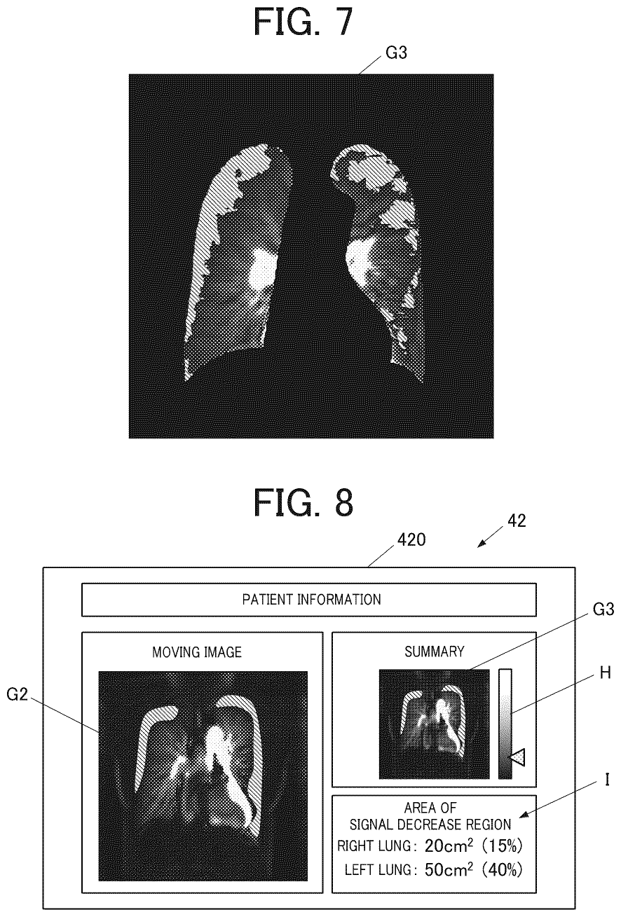

FIG. 7 is a diagram illustrating an example of the dynamic analysis image G3 after the signal decrease region is emphasized according to the present embodiment. In FIG. 7, the yellow-green signal decrease region is indicated by hatching for convenience. In the dynamic analysis image G3, the peripheral parts of the right lung and the left lung in the lung field region are mainly emphasized in yellowish green as the signal decrease region. Thus, a user, such as a doctor, can accurately and quickly confirm the region where a blood flow defect occurs in the lung field region of the dynamic analysis image G3.

The controller 40 calculates the area of the signal decrease region in the processing region of the dynamic analysis image G3 (step S7). For example, the controller 40 calculates the ratio of the area of the signal decrease region to the entire area of the processing region of the dynamic image analysis. After calculating the area of the signal decrease region, the controller 40 proceeds to step S8.

The controller 40 determines whether the calculated area as the quantitative value of the signal decrease region is equal to or greater than the second threshold (step S8). Step S8 corresponds to a determination step. The second threshold can be set to, for example, 20%. In general, the possibility of a blood flow defect increases when the ratio of the area of the signal decrease region to the entire area of the processing region in the dynamic analysis image G3 is 20% or greater. The second threshold is not limited to 20% and can be appropriately changed depending on the purpose of the inspection. For example, since a screening inspection requires a higher level of sensitivity, the second threshold may be set to a smaller value.

When the controller 40 determines that the calculated areal ratio, which is the ratio of the area of the signal decrease region to the area of the entire processing region of the dynamic analysis image G3, is equal to or greater than the second threshold, the controller 40 proceeds to step S9. In this case, the controller 40 determines that a blood flow defect occurs in the processing region of the acquired dynamic analysis image G3 (step S9) and proceeds to step S10. At this time, in a case where there is no abnormality in the background lung in the processing region of the dynamic analysis image G3, the controller 40 may determine that there is a possibility of pulmonary embolism. On the other hand, when there is an abnormality in the background lung in the processing region of the dynamic analysis image G3, the controller 40 may determine that there is a possibility of a disease other than pulmonary embolism. When determining in step S8 that the calculated area of the signal decrease region in the processing region is less than the second threshold, the controller 40 proceeds to step S12. In this case, the controller 40 determines that there is no blood flow defect in the predetermined region of the acquired dynamic analysis image G3 (step S12) and proceeds to step S10.

The controller 40 outputs, to the display part 42, the PH2-mode dynamic analysis image G2, the dynamic analysis image G3 indicating the signal decrease region, and the determination information on a blood flow defect, and so forth (step S10). The determination information includes, for example, statistical information I of the signal decrease region. The determination information may also include information on whether a blood flow defect is present, a disease name, such as a pulmonary embolism, and so forth.

FIG. 8 is a diagram illustrating an example of the PH2-mode dynamic analysis image G2, the dynamic analysis image G3 in which the signal decrease region is emphasized, and the statistical information I that are displayed on the display part 42 according to the present embodiment. The inspection screen 240 of the display part 42 displays the PH2-mode dynamic analysis image G2, the dynamic analysis image G3 generated from the dynamic analysis image G2, and the statistical information I regarding the signal decrease region. The dynamic analysis image G2 is a dynamic image after PH2-mode gain adjustment is performed and is displayed on the left side of the screen, for example. The dynamic analysis image G3 is a still image in which the signal decrease region corresponding to a low blood flow in the lung field region is highlighted. The dynamic analysis image G3 is displayed on the right side of the screen, for example. The dynamic analysis image G3 is not limited to a still image and may be a moving image. In the dynamic analysis image G3, a threshold adjustment slider H for adjusting the first threshold is provided. With the threshold value adjustment slider H, it is possible to change the sensitivity regarding the signal decrease region. The statistical information I is displayed, for example, below the dynamic analysis image G3. In the statistical information I, for example, the area of the signal decrease region and the occupancy rate thereof are displayed for the right lung and the left lung of the lung field region in the dynamic analysis image G3. The statistical information I may include determination information indicating whether a blood flow defect is present, the disease name such as pulmonary embolism, and so forth.

As the display content of the display part 42, at least one of the PH2-mode dynamic analysis image G2, the dynamic analysis image G3 in which the signal decrease region is highlighted, and the statistical information I may be displayed. The display of the signal decrease region in the dynamic analysis image G3 may be turned on and off. For another example, a plurality of dynamic analysis images G3 including the signal decrease regions determined using a plurality of first thresholds may be displayed side by side on the display part 42. The first threshold may not be adjusted with the threshold adjustment slider H. For example, the first threshold may be adjustable by directly inputting a numerical value or manipulating a numerical value increase-decrease button.

In the above embodiment, the first threshold value is set, based on the tones (stages) of color display when pixels of the lung field region in the dynamic analysis image G2 after gain adjustment are displayed in color. However, the present invention is not limited thereto. For example, the first threshold may be set, based on the X-ray transmission amount of pixels of the lung field region in the dynamic analysis image on which gain adjustment is not performed. Specifically, the first threshold can be set as follows. Generally, the pulsation of blood vessels changes by about 6% in a site where the pulsation of blood vessels is sufficiently performed, for example, the pulmonary artery origin. When the change in the pulsation of blood vessels falls below 0.9% with respect to about 6%, the possibility of a blood flow deficiency increases. Therefore, the first threshold can be set to 0.9%. The change in the pulsation of blood vessels, which is about 6%, may be expressed as 100. In this case, the first threshold value may be set to 15 since “0.9” corresponds to 15% of “6”.

The method of determining whether a blood flow defect occurs from the dynamic analysis image G3 is not limited to the above-described method. Although the presence of a blood flow defect is determined using the area of the signal decrease region in the above-described example, it may be determined using the area of the non-signal decrease region, for example. In this case, the second threshold can be set to less than 80%, for example. When the area of the non-signal decrease region is less than 80%, the controller 40 can determine that a blood flow defect occurs in the signal decrease region. As another determination method, for example, a ratio between the signal decrease region and the non-signal decrease region may be used. The ratio of the signal decrease region to the non-signal decrease region is, for example, 1:4. In this case, when the ratio of the signal decrease region to the non-signal decrease region is 1:4 (¼ or more), the controller 40 determines that a blood flow defect occurs. For another example, the presence or absence of the occurrence of the blood flow defect may be determined using a value obtained by dividing the ratio of the signal decrease region by the ratio of the non-signal decrease region. In this case, the second threshold is set to, for example, 0.25 or more.

According to the present embodiment, the dynamic analysis image G3 indicating the signal decrease region and the statistical information I on a blood flow defect are displayed on the display part 42. Therefore, even a doctor, such as a practitioner or a resident unskilled in interpreting the PH2-mode dynamic analysis image G2 and so forth, can intuitively evaluate a blood flow defect. Accordingly, the doctor can diagnose the patient without overlooking a disease. Furthermore, according to the present embodiment, the area of the signal decrease region and/or the occupancy rate of the signal decrease region in the dynamic analysis image G3 are displayed on the display part 42. Therefore, the doctor is allowed to determine whether a disease is present objectively. For example, it is possible to determine whether a blood flow defect is getting better or worse by comparing the current area of the signal decrease region with the area of the signal decrease region in the past dynamic analysis image.

### First Modification Example

In the above-described embodiment, the signal decrease region in the processing region is determined using one first threshold. Instead, a plurality of first thresholds may be set according to the distance from a reference position, such as the center of the processing region. In general, the blood flow rate of the pulmonary artery decreases from the center of the large blood vessel toward the periphery before the capillaries. A blood vessel close to the heart is thicker and flows more blood. A blood vessel far from the heart is thinner and flows less blood. Therefore, in some cases, the signal decrease region can be determined with high accuracy by setting first thresholds in consideration of the original blood flow in a state where no disease, such as a blood flow defect, is present. The first threshold may be changed depending on the posture in imaging. For example, in the standing imaging, the first threshold may be set according to the imaging position in the vertical direction. The first threshold is increased for a lower position and decreased for an upper position. This is because in the standing posture, the blood flow on the lower side increases, whereas the blood flow on the upper side decreases, due to the influence of gravity.

In the first modification example, the lung field region of the dynamic analysis image G2 is divided into multiple concentric regions with the center of the lung field region as a reference; and different first thresholds are set for the respective divided regions, for example. The components having common configurations and functions with the above-described embodiment will be denoted by the same reference numerals, and detailed description thereof will be omitted.

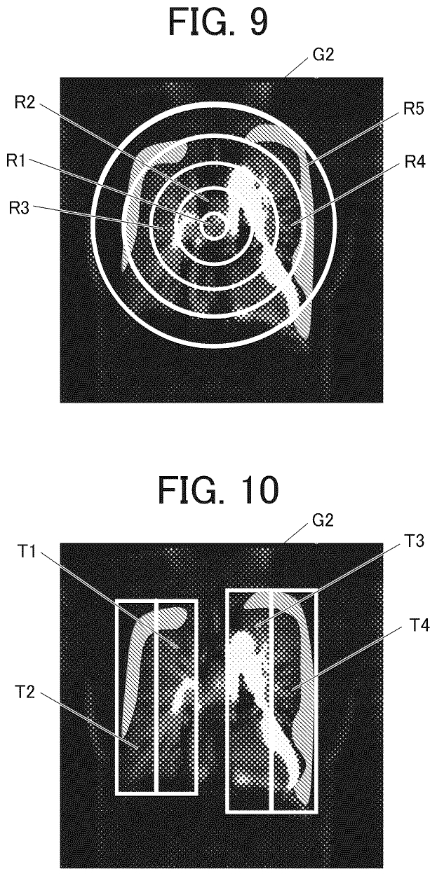

FIG. 9 is a diagram illustrating an example of a method of setting first thresholds and so forth according to the first modification example. In the first modification example, the lung field region of the dynamic analysis image G2 is concentrically divided into a first region R1, a second region R2, a third region R3, a fourth region R4, and a fifth region R5 from the center of the lung field region toward the outside. The heart is located substantially in the center of the lung field region. The first region R1 is closest to the center of the lung field region, and the fifth region R5 is farthest from the center of the lung field region. Next, different first thresholds are set for the respective divided first region R1, second region R2, third region R3, fourth region R4, and fifth region R5. Specifically, the farther the region is from the center of the lung field region, the smaller first threshold is set. That is, the first thresholds are set to become smaller in the order of the first region R1, the second region R2, the third region R3, the fourth region R4, and the fifth region R5.

For each of the divided regions of the lung field region in the dynamic analysis image, the controller 40 determines whether the pixels in each region correspond to the signal decrease region, based on the first threshold of each region. For example, the controller 40 determines whether each pixel in the first region R1 of the lung field region is equal to or less than the first threshold set for the first region R1. Similarly, the controller 40 determines whether pixels in the respective second region R2, third region R3, fourth region R4, and fifth region R5 corresponds to the signal decrease region, based on the first thresholds set for the respective regions.

Further, when the lung field region of the dynamic analysis image G2 is divided into a plurality of concentric regions with the center of the lung field region as a reference, the second threshold values may be set for the respective divided regions. Specifically, the second thresholds are set for the respective first region R1, second region R2, third region R3, fourth region R4, and fifth region R5 illustrated in FIG. 9. The second thresholds can be set, based on the ratio of the area of the signal decrease region in the divided region to the entire area of the divided region in a dynamic analysis image that is obtained by the other dynamic imaging. The second threshold can be arbitrarily set. For example, the second threshold can be 20%.

The controller 40 calculates the signal decrease region in each of the first region R1, the second region R2, the third region R3, the fourth region R4, and the fifth region R5 into which the lung field region of the dynamic analysis image G2 is divided. The controller 40 then determines whether the calculated area of the signal decrease region in each region is equal to or greater than the second threshold. Specifically, the controller 40 determines whether the area of the signal decrease region in the first region R1 is equal to or greater than the second threshold set for the first region R1. Similarly, the controller 40 determines whether the area of the signal decrease region in each of the second region R2, the third region R3, the fourth region R4, and the fifth region R5 is equal to or greater than the second threshold set for each of the regions.

According to the first modification example, the lung field region in the dynamic analysis image is divided into a plurality of regions according to the distance from the reference position, such as the heart; and the first threshold and the second threshold are set for each of the divided regions. Thus, it is possible to determine whether a blood flow defect is present (whether the respective regions correspond to the signal decrease region), in consideration of the original blood flow in a state where there is no disease such as the blood flow defect. As a result, it is possible to prevent misdiagnosis in which a region originally having low blood flows is regarded as the signal decrease region corresponding to a blood flow defect.

### Second Modification Example

In the second modification example, multiple regions are provided for each of the left lung and the right lung of the lung field region with the center of the lung field region as a reference; and for the respective regions, different first thresholds are set. The components having common configurations and functions with the above-described embodiment will be denoted by the same reference numerals, and detailed description thereof will be omitted.

FIG. 10 is a diagram illustrating an example of a method for setting the first thresholds and so forth according to the second modification example. In the second modification example, the right lung of the lung field region in the dynamic analysis image G2 is divided into a first region T1 in the center side and a second region T2 in the outer side. Similarly, the left lung of the lung field region in the dynamic analysis image G2 is divided into a third region T3 in the center side and a fourth region T4 in the outer side. The first region T1 and the third region T3 are closest to the center of the lung field region, and the second region R2 and the fourth region T4 are farthest from the center of the lung field region. In the present embodiment, different first thresholds are set for the divided first region T1, second region T2, third region T3, and fourth region T4. Herein, the farther the region is from the center of the lung field region, the smaller first threshold is. Since the first region T1 and the third region T3 are far from the center of the lung field region by a substantially equal distance, they may have the same first threshold. Similarly, since the second region T2 and the fourth region T4 are far from the center of the lung field region by a substantially equal distance, they may have the same first threshold.

For each of the divided regions of the lung field region in the dynamic analysis image, the controller 40 determines whether the pixels in each region correspond to the signal decrease region, based on the first threshold determined for each region. For example, the controller 40 determines whether the pixels in the first region R1 of the lung field region are equal to or less than the first threshold set for the first region T1. Similarly, the controller 40 determines whether the pixels in the second region T2, the third region T3, and the fourth region T4, respectively, correspond to the signal decrease region, based on the first thresholds set for the respective regions.

Further, when the left lung and the right lung of the lung field region in the dynamic analysis image G2 are divided into a plurality of regions with the center of the lung field region as a reference, the second threshold values may be set for the respective divided regions. Specifically, the second thresholds are set for the first region R1, the second region R2, the third region R3, and the fourth region R4, respectively, as illustrated in FIG. 10. The second thresholds can be set, based on the ratio of the area of the signal decrease region in the divided region to the entire area of the divided region in a dynamic analysis image that is obtained by the other dynamic imaging. The second threshold can be arbitrarily set. For example, the second threshold can be 20%.

The controller 40 calculates the signal decrease region in each of the first region R1, the second region R2, the third region R3, and the fourth region R4 into which the lung field region of the dynamic analysis image G2 is divided. The controller 40 then determines whether the calculated area of the signal decrease region in each region is equal to or greater than the second threshold. Specifically, the controller 40 determines whether the area of the signal decrease region in the first region R1 is equal to or greater than the second threshold set for the first region R1. Similarly, the controller 40 determines whether the area of the signal decrease region in each of the second region R2, the third region R3, and the fourth region R4 is equal to or greater than the second threshold set for each of the regions.

According to the second modification example, the lung field region in the dynamic analysis image is divided into a plurality of regions according to the distance from the reference position, such as the heart; and the first threshold and the second threshold are set for each of the divided regions. Thus, it is possible to determine whether a blood flow defect is present (whether the respective regions correspond to the signal decrease region), in consideration of the original blood flow in a state where there is no disease such as the blood flow defect. As a result, it is possible to prevent misdiagnosis in which a region originally having a low blood flow is regarded as the signal decrease region corresponding to a blood flow defect.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

Specifically, in the above-described embodiment, gain adjustment is performed on the generated dynamic analysis image; after the gain adjustment, the signal decrease region in the processing region is detected; and it is determined whether a blood flow defect is present. However, the present invention is not limited thereto. For example, it is also possible to detect a signal decrease region in the processing region of the dynamic analysis image on which gain adjustment is not performed, and to determine whether a blood flow deficiency occurs.

In the above-described embodiment, the areas of the signal decrease region and the non-signal decrease region are used to determine whether a blood flow defect occurs in the dynamic analysis image G3. However, the present invention is not limited thereto. For example, signal values in the signal decrease region and the signal non-decrease region of the dynamic analysis image G3 may be measured; and the average value, the standard deviation, and the integral value may be calculated from the measured signal values. The controller 40 can determine whether a blood flow defect occurs by comparing the quantitative values obtained by calculations and a second threshold, which is set beforehand based on quantitative values.

Although embodiments of the present invention have been described and shown in detail, the disclosed embodiments are made for purposes of illustration and example only and not limitation. The scope of the present invention should be interpreted by terms of the appended claims.

What is claimed is:

1. A dynamic analysis apparatus configured to perform dynamic analysis on a dynamic image obtained by irradiating a subject with radiation, the dynamic analysis apparatus comprising a hardware processor, wherein:
the hardware processor generates a dynamic analysis image for measuring a blood flow, based on signal values of pixels constituting the dynamic image,
when a pixel of the dynamic analysis image has a signal value less than or equal to a first threshold, the hardware processor detects the pixel as a signal decrease region that indicates a decrease in a blood flow value, and
the hardware processor compares a quantitative value of the detected signal decrease region or a quantitative value of a non-signal decrease region with a second threshold, the non-signal decrease region being other than the signal decrease region, and based on the comparison, determines whether a blood flow defect is present.

2. The dynamic analysis apparatus according to claim 1, wherein:
the quantitative value is an area, and
the second threshold is a ratio between an area of the signal decrease region and an area of an entire processing region in the dynamic image.

3. The dynamic analysis apparatus according to claim 1, wherein:
a processing region in the dynamic image is divided into multiple divided regions,
the quantitative value is an area, and
the second threshold value is set for each of the divided regions, based on a ratio between an area of the signal decrease region in the divided region and an area of the entire divided region.

4. The dynamic analysis apparatus according to claim 1, wherein:
the dynamic analysis image is expressed in multiple tones of color according to the signal values of the pixels constituting the dynamic analysis image, and
the first threshold is set to a predetermined tone of color among the multiple tones, the predetermined tone corresponding to a blood flow defect.

5. The dynamic analysis apparatus according to claim 4, wherein:
the hardware processor corrects a variation in the generated dynamic analysis image caused by different imaging conditions in imaging, and
the first threshold is set, based on the dynamic analysis image on which the correction of the variation is performed.

6. The dynamic analysis apparatus according to claim 1, wherein
a processing region in the dynamic image is divided into multiple divided regions according to a distance from a predetermined reference position, and
different first thresholds are set to the respective divided regions.

7. The dynamic analysis apparatus according to claim 1, wherein
when determining that a blood flow defect is present in the dynamic analysis image, the hardware processor outputs determination information on the blood flow defect.

8. The dynamic analysis apparatus according to claim 7, wherein the hardware processor colors the signal decrease region in the dynamic analysis image with a color different from a color of the non-signal decrease region and outputs the colored dynamic analysis image.

9. A dynamic analysis method of performing dynamic analysis on a dynamic image obtained by irradiating a subject with radiation, the method comprising:
generating a dynamic analysis image for measuring a blood flow, based on signal values of pixels constituting the dynamic image;
when a pixel of the dynamic analysis image has a signal value less than or equal to a first threshold, detecting the pixel as a signal decrease region that indicates a decrease in a blood flow value; and
comparing a quantitative value of the detected signal decrease region or a quantitative value of a non-signal decrease region with a second threshold, the non-signal decrease region being other than the signal decrease region, and based on the comparison, determining whether a blood flow defect is present.

10. A non-transitory computer-readable storage medium storing a program for a computer of a dynamic analysis apparatus configured to perform dynamic analysis on a dynamic image obtained by irradiating a subject with radiation, the program causing the computer to:
generate a dynamic analysis image for measuring a blood flow, based on signal values of pixels constituting the dynamic image,
when a pixel of the dynamic analysis image has a signal value less than or equal to a first threshold, detect the pixel as a signal decrease region that indicates a decrease in a blood flow value, and
compare a quantitative value of the detected signal decrease region or a quantitative value of a non-signal decrease region with a second threshold, the non-signal decrease region being other than the signal decrease region, and based on the comparison, determine whether a blood flow defect is present.
